(19) United States
(12) Patent Application Publication
(10) Pub. No.: US 2006/0035846 A1
(43) Pub. Date: Feb. 16, 2006

(54) Preventing/correcting functional disorders of the pilosebaceous unit of mammals

(75) Inventors: Albert Duranton, Maisons-Lafitte (FR); Armand Malnoe, Montaubion Chardonney (CH)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignees: L'OREAL, PARIS (FR); NESTEC S.A., VEVEY (CH)

(21) Appl. No.: 11/107,866

(22) Filed: Apr. 18, 2005

Related U.S. Application Data
(63) Continuation of application No. PCT/EP03/14777, filed on Oct. 16, 2003.
(60) Provisional application No. 60/441,762, filed on Jun. 3, 2003.

(30) Foreign Application Priority Data
Oct. 16, 2002 (FR) 02/12887

(51) Int. Cl. A61K 31/7048; A61K 31/353; A61K 31/202; A61K 31/05

(52) U.S. Cl. 514/27; 514/456; 514/560; 514/733

(57) ABSTRACT

Orally administratable cosmetic compositions, notably formulated as food supplements, useful for the prevention and/or treatment of functional disorders of the pilosebaceous structural unit of mammals, in particular by combating the cutaneous activation of the androgens over time, and especially for the treatment of oily and/or hyper-seborrheic skin and/or scalp, thin hair, hypertrichosis and/or alopecia in humans, comprise thus effective amounts of immixture of polyunsaturated fatty acids, flavonoids and stilbenes, formulated into orally administratable, physiologically acceptable media therefor.

### CROSS-REFERENCE TO PRIORITY/PCT/PROVISIONAL APPLICATIONS

This application claims priority under 35 U.S.C. § 119 of FR 02/12887, filed Oct. 16, 2002, and of provisional application Ser. No. 60/441,762, filed Jan. 23, 2003, and is a continuation of PCT/EP 2003/014777, filed Oct. 16, 2003 and designating the United States (published on Apr. 29, 2004 in the English language as WO 2004/034820 A3), each hereby expressly incorporated by reference and each assigned to the assignee hereof.

### BACKGROUND OF THE INVENTION

1. Technical Field of the Invention

The present invention relates to novel compositions and treatment procedures for cosmetic purposes suited for human and animals. In particular, the present invention relates to cosmetic products which can be administered by the oral route, such as food supplements.

Thus, this invention relates to cosmetic compositions suitable for administration by the oral route and useful to prevent and/or correct the functional disorders of the pilosebaceous unit of mammals, in particular by combating the cutaneous activation of the androgens over the course of time, said compositions comprising polyunsaturated fatty acids, flavonoids and stilbenes, formulated into a physiologically acceptable medium therefor.

The present invention also relates to a cosmetic method (regime or regimen) employing such compositions to prevent and/or correct the functional disorders of the pilosebaceous unit of mammals.

2. Description of Background and/or Related and/or Prior Art

The hairs of mammals in general, the hairs and hair of humans in particular, are keratinized filaments of variable length.

In the remainder of the text the term “hair” will designate both body hairs of humans and animals, and the hair of humans. Consequently, the adjective “hairy” will refer to hairs as such or to hair or also to both. In every case, the sense to be given to this adjective will emerge in a clear and unambiguous manner from the text.

In the sense of the present invention, unless otherwise stated the term “human” is meant to designate both man and woman.

The hairs comprise a fixed part or root, implanted in an invagination of the epidermis, the hairy follicle and a free part or stem, more or less tapering at its extremity. The root of the hair adheres to the hairy follicle and forms, at its base, a bulb emptied at its center where is lodged the dermal papilla rich in capillaries with a nutritive function.

At the base of each hair a sebaceous gland is appended. This latter secretes a semi-liquid fatty substance, the sebum, a mixture of fatty acids, triglycerides, cholesterol, cholesterol derivatives and cell debris (Stewart, M. E. Semin Dermatol 11, 100-105 (1992)). In humans, for example, the sebaceous glands cover the whole of the body with the exclusion of the palms of the hands and the soles of the feet. Besides the scalp, these glands are particularly widespread around the nose, the mouth, on the forehead, the cheek, the chin and the neck, where their density may vary between about 400 and 900 glands per square centimeter.

The sebum, secreted by the sebaceous gland at the level of the hairy follicle, lubricates the surface of the skin, the hairs and the hair. It forms a slightly acidic fatty film, designed to protect the skin against external aggressions and dehydration, and does so due to its antiseptic properties, its “barrier” function towards the exterior and its capacity to retain water.

However, when the sebum is secreted in excessive quantities, the skin, the hairs and the hair assumes a glossy sheen which can reveal itself as particularly unaesthetic. One then speaks of “impairments”, “disorders”, “disturbances” or functional disorders of the pilosebaceous unit.

In the present context, it is understood that the terms pilosebaceous “unit”, “organ” and “apparatus” can be used interchangeably to designate the complete structure constituted by the hairy follicles and the associated sebaceous glands.

The disorders mentioned above may sometimes reveal themselves to be severe, a simple sebaceous hypersecretion or seborrhea, capable of developing to quite extensive forms of alopecia.

In mammals, the development and function of the sebaceous glands are regulated by complex mechanisms involving the endocrine system (Mercurio et al.; J. Gend Specif Med, 2000 May-June; 3 (4): 59-64).

The endocrine system groups together the endocrine glands, the products of secretion of which, the hormones, are discharged directly into the blood.

Chemical messengers of the organism, the hormones thus produced usually act on an organ or a specific target tissue.

The hormones do not constitute a homogeneous chemical class. In fact, under this generic term, are grouped together compounds of structure as different as proteins, peptides, steroids, aromatic compounds and amines.

In particular, the sex hormones such as testosterone, the estrogens or progesterone belong to the family of the steroids, hormonal compounds derived from cholesterol. These include in particular the male sex hormones, the androgens.

It has been observed that the pilary density of mammals is modified over the course of time.

As regards the hair, the number and diameter of the capillary stems diminish with age. Certain masculine subjects even go so far as to develop andro-genetic alopecias. Furthermore, these adverse changes of the hair are sometimes accompanied by a modification of the state of the scalp, such as an overabundant production of sebum.

In women, age may cause to appear anomalies of pilosity, for example an excess of hairs on the legs, the arms or the growth of hairs at the level of the beard or the moustache.

Studies have shown that active hormones, in particular sex steroids, are formed by conversion of inactive precursors in the peripheral target tissues, such as the mammary glands, the prostate, the uterus, the bones and the skin. Thus, the androgenic and estrogenic hormones exert an activity in these tissues.

As a result, the adverse changes in the pilosebaceous unit mentioned above are due essentially to an abnormal metabolism of the steroid hormones, in particular of the androgens (Labrie et al.; Horm Res 2000; 54(5-6): 218-29). More exactly, said adverse changes are the result of a local activation, i.e., cutaneous, of the androgens, and in particular of testosterone during the course of time (Labrie et al; Horm Res 2000; 54 (5-6): 218-29; Example 1, infra).

At present, the functional disorders of the pilosebaceous apparatus of mammals remain difficult to treat from a cosmetic point of view. On the one hand, the topical route proves to be poorly adapted in as much as the surfaces to be treated are most often large and require an at least daily frequency of treatment On the other hand, the products likely to be administered by the oral route and which are currently available on the market exhibit non-negligible side effects, especially sexual. Their use consequently requires a regular control and close supervision by medically qualified personnel.

Consequently, need continues to exist for agents active against the above-mentioned disturbances, said active agents being adapted for administration by the oral route and devoid of side effects.

### SUMMARY OF THE INVENTION

The present invention satisfies the above objectives.

Thus, the present invention features cosmetic compositions useful for the prevention and/or the correction of the functional disorders of the pilosebaceous unit of mammals, these compositions being designed for administration via the oral route and comprising intimate immixture of polyunsaturated fatty acids, flavonoids and stilbenes.

Such compositions make it possible to combat the cutaneous activation of the androgens, including testosterone and the adrenalin androgens, over the course of time.

The compositions according to the invention are especially useful for the treatment of oily and/or hyper-seborrheic skin and/or scalp, thin hair, hypertrichosis or excess of hairs and/or alopecia, in humans.

### DETAILED DESCRIPTION OF BEST MODE AND SPECIFIC/PREFERRED EMBODIMENTS OF THE INVENTION

More particularly according to the present invention, the subject compositions are preferably food or nutritional supplements.

In the context of the present invention, the term “mammal” must be understood in conformity with the general usual definition. This notwithstanding, the term “mammals” in the sense of the invention advantageously targets domestic animals and/or human beings.

In a preferred embodiment, the polyunsaturated fatty acids and/or the flavonoids and/or the stilbenes contained in the compositions according to the invention are of natural origin. In a still more preferred embodiment, the set of these compounds is of natural origin. By “natural origin” is meant a compound in the pure state or in solution at variable concentrations, said compound being obtained by various extraction processes known to this art, starting from a natural element, for example a plant or an animal. More preferably, all the compounds are extracted from materials which are usually used as food.

The polyunsaturated fatty acids are essentially polyethylenic acids, i.e., comprising at least two carbon-carbon double bonds. In fact, there are in nature few polyunsaturated fatty acids containing carbon-carbon triple bonds.

According to a first embodiment of the compositions of the invention, the polyunsaturated fatty acids are essential fatty acids. These fatty acids, although they cannot be synthesized by the mammals themselves, are essential to the normal metabolism of said mammals (growth, development and integrity of the skin, reproductive functions, in particular gestation and lactation . . . ). The essential fatty acids must be taken with the food.

The polyunsaturated fatty acids originating in foods are defined by the length of the carbon chain and the position of the double bonds. Thus, they are classed in two groups, without metabolic conversion nor functional substitution being possible from one to the other, said groups being distinguished by the position of the unsaturation closest to the terminal methyl group. These groups are conventionally designated n-3 (or ω3 or “omega-3”) and n-6 (or ω6 or “omega-6”), As examples, fatty acids of group n-3 are α-linolenic acid [18 (number of carbon atoms): 3 (number of unsaturations) n-3] and stearidonic acid (18:4 n-3). Fatty acids of group n-6 are, for example, linoleic acid (18:2 n-6) and γ-linolenic acid (18:3 n-6).

Of the essential fatty acids suitable for the formulation of a composition according to the invention, one skilled in this art will preferably select polyunsaturated fatty acids belonging to group n-6. In particular, γ-linolenic acid will be advantageously selected.

As indicated above, the fatty acids contained in a composition in conformity with the invention are preferably extracts of animal or plant natural sources. In this respect, black currant pip oil, evening primrose oil; borage and hemp may be mentioned as oils of plant origin, the extracts of spirulin, in particular Spirula maxima and S. platensis, and the oils of fishes and those derived from sea products as oils of animal origin. Obviously, these oils can be used alone or in the form of mixtures. Preferably, one skilled in this art will select the black currant pip oil as source of polyunsaturated fatty acids.

According to another embodiment of a composition of the invention, the latter contains flavonoids, in particular isoflavonoids.

The flavonoids are polyphenols which, depending on their structure and the degree of oxidation to which they are subjected, are divided into flavonoids, flavones, catechins, proanthocyanidines and their analogues and derivatives.

The natural sources of flavonoids are mainly plants, in particular the representatives of the families of the Umbelliferae, Rosaceae, Leguminosae and other compounds. Flavonoids are also found in products derived from vines and tea.

The isoflavonoids constitute a sub-class of the flavonoids. They are formed of a 3-phenylchromane skeleton which can bear varied substituents at different levels of oxidation. Contrary to the flavonoids, the isoflavonoids are present in only a very limited number of plants.

In fact, the term “isoflavonoid” designates a set of classes of compounds, including the isoflavones, the isoflavanones, the rotenoids, the pterocarpans, the isoflavanes, the isoflavan-3-enes, the 3-arylcoumarins, the 3-aryl-4-hydroxycoumarins, the coumestanes, the coumaronochromones, the α-methyldesoxybenzoins or the 2-arylbenzofurans. For a complete review of the isoflavonoids, the methods of their analysis and their sources, see Chapter 5 entitled “Isoflavonoids” of the monograph “The Flavonoids” (Dewick, P. M. Harbone Ed. pp. 125-157 (1988)).

Isoflavonoids particularly suited according to the present invention are, for example, daidzein, formononetin, cuneatin, genistein, isoprunetin and prunetin, cajanin, orobol, pratensein, santal, junipegenin A, glyciteiri, afrormosin, retusin, tectorigenin, irisolidon, jamaicin as well as their analogues and derivatives.

Of the isoflavonoids used in the context of the invention, preference will be given to the isoflavones, and in an even more preferred embodiment to the simplest aglycone forms, including daidzein, genistein and their mixture. These two compounds are particularly present in the extract of soja Glycina max.

According to another specific embodiment of the invention, two different sources of extracts which are rich in flavonoids are used, one rich in procyanidines and another one rich in isoflavonoids.

According to another embodiment of a composition of the invention, the latter contains stilbenes, in particularly hydroxystilbenes.

The stilbenes, possibly glycosylated, are produced by plants, essentially the spermatophytes, and belong to the class of antibiotic molecules called phytoalexines. Of these, mention may be made of resveratrol or 3,5,4′-trihydroxystilbene.

Resveratrol is produced by many fruits and plants, in a simple (trihydroxystilbene) or glycosylated form (piceid, polydatin or 4′,5-dihydroxystilbene-3-O-β-mono-D-glucoside, for example). Simple and glycosylated forms of resveratrol are particularly present in the skin of the grape (Vrhovsek et al., Am J Enol Vitic, 48 (2) (1997)), or also in in vitro culture supernatants of Vitis vinifara (Teguo et al., J Nat Prod, 61, 655-657 (1998)).

Cosmetic compositions comprising resveratrol have already been described in the context of the control of signs of skin aging (WO 99/04747 in the name of Unilever, N. V.). Similarly, a procedure has been described for obtaining ester derivatives of resveratrol and the use of these latter in cosmetic compositions as precursors of resveratrol (WO 99/03816 in the name of Caudalie). Finally, the assignee hereof has described the advantageous use of glycosylated hydroxylstilbene derivatives, more stable and more soluble than the corresponding hydroxystilbenes, as precursors of these latter for the preparation of cosmetic, dermatological or pharmaceutical compositions designed for a topical application (French patent application No. 0010008 Filed on 28 Jul. 2000).

The hydroxystilbenes may be extracts of plants or plant materials containing them which include the following plant families, cited purely as illustrations: Vitaceae, Ombelliferae, Mvrtaceae, Dipterocarpaceae, yperaceae, Gnetaceae, Leguminosae, Graminaeae, Sericeae, Haemodoraceae, Muaceae, Polygonaceae, Pinaceae, Crupressaceae, Cesalpiniaceae, Poaceae, Solanaceae. In particular, they can be isolated from tissues of V. vinifera or Polygonum cuspidatum. The skilled artisan will select grape skin or products derived from the grape such as wine as preferred sources of hydroxystilbenes. For example, grape extracts can be obtained from the marc of grapes, the pips and/or seed envelopes and/or possibly the stalks of grapes.

Thus, in the present context, extracts of red wine or grape pips, rich in polyphenolic compounds of the resveratrol type and proanthocyanidines may advantageously be used.

According to one embodiment of a composition of the invention, the latter is packaged in the form of unit doses adapted to administration by the oral route, said administration being carried out at the rate of 1 to 6 doses or units per day. Preferably, the dose of 3 units/day is recommended.

The daily doses recommended in conformity with the invention range from 0.5 to 2600 mg/day, and preferably from 5 to 1200 mg/day of polyunsaturated fatty acids, from 0.5 to 1000 mg/day, and preferably from 20 to 300 mg/day of flavonoids, and from 0.5 to 1000 mg/day, and preferably from 10 to 200 mg/day of stilbenes.

The compositions of the invention can be taken for several days, weeks or months. The regimen of treatment can be repeated many times in a year and can be even continuous. Indeed, the active extracts used in the compositions originates from usual food sources for human or animal, and does not lead to risks of toxicity.

These recommended daily doses are such that a composition according to the invention comprises admixture of the following quantities in weight per 1 g dose unit:
from 0.1% to 99%, preferably from 5% to 60% and still more preferred from 10% to 40% of polyunsaturated fatty acids;
from 0.1% to 99%, preferably from 5% to 60% and still more preferred from 10% to 30% of flavonoids; and more preferably from 20% to 30%;
from 0.1% to 99%, preferably from 5% to 40% and still more preferred from 10% to 25% of stilbenes.

The compositions according to the invention may be presented in all possible imaginable galenical forms, provided that these are adapted to administration by the oral route. Thus, there may be mentioned as non-limiting examples, a drinkable solution, a syrup, a tablet, a coated tablet, a gelatine capsule, or even an enriched foodstuff such as a biscuit, a nutrient bar or powder, possibly compacted. The powders may be diluted in water, soda, milk products or soja derivatives or be incorporated into bars or biscuits, for example. Preferably, the composition according to the invention is a solid composition, and for example, a tablet, a coated tablet, a gelatine capsule, or even an enriched foodstuff such as a biscuit, a nutrient bar or powder, possibly compacted.

According to another embodiment of a composition in conformity with the invention, the latter comprises, in addition to polyunsaturated fatty acids, flavonoids and stilbenes, at least one excipient appropriate for an oral administration. In one specific embodiment, the composition of the invention comprises polyunsaturated fatty acids, flavonoids stilbenes, and excipients appropriate for an oral administration. In this respect, the formulation agents, adjuvants and excipients for oral compositions, in particular for foodstuff supplements are known to the skilled artisan. Among others and in a purely illustrative manner, mention may be made of lubricants such as magnesium stearate, products for instantaneous solubilization, gelling agents, thickeners, moisteners, fatty and/or aqueous compounds, preservatives, texturizing, flavoring and/or glazing agent, anti-oxidants and coloring agents usual in the food sector.

The compositions according to the present invention may in addition contain one or more vitamins and/or trace elements. As a guide, the following active compounds may, for example, be used alone or in combination: zinc and its salts including zinc sulfate and zinc glucanate, the vitamins B5, B6, B8, C, E or PP, β-carotene and the carotenoids, garlic extracts particularly in the form of allyl sulfide or ajoen, selenium, curcurmine, the curcuminoids, niacin, lithospermic acid and adenosine. It is understood that one skilled in this art will select such active compounds and, if necessary, will combine them so as to improve the effects expected of the composition which is the object of the invention, and avoid the inhibition or attenuation of the desired activity of interest.

In one specific embodiment, the composition does not contain any Lotus (Nelumbo) extract, and especially, the composition does not contain any active substance of the Lotus, selected among methyltransferases, ascorbic acid, gluthatione and dopamine agonists.

In another specific embodiment, the composition does not contain any DHEA or an analogue thereof.

In another specific embodiment, the composition of the invention does not contain panthetin.

In another specific embodiment, the composition of the invention does not contain milk or compounds extracted from milk.

The present invention also features a cosmetic method (regime or regimen) for the prevention and/or the correction of the functional disorders of the pilosebaceous unit of mammals, particularly of human beings and domestic animals, and more specifically for the treatment of oily and/or hyper-seborrheic skin and/or scalp, thin hair, hypertrichosis or excess of hairs and/or alopecia, said process entailing administering by the oral route a composition as described above. More preferably, the cosmetic method is applied to humans.

Finally, the present invention also features the use of a composition as referred to above, containing polyunsaturated fatty acids, flavonoids and stilbenes for the preparation of a foodstuff supplement designed to prevent and/or correct the functional disorders of the pilosebaceous unit of mammals, in particularly of human beings and domestic animals.

Thus, this invention features the use of a composition as above-defined in a cosmetic regime or regimen suited to increase the density of keratinic fibers and especially hair, and/or reduce the heterogeneity of their diameter and/or improve their growth, and/or prevent and/or reduce and/or delay hair loss.

As used herein, the term “heterogeneity of hair diameters” refers to a significant variation in the hair diameter in a specific region of the scalp; some hair having a physiological diameter in the range of 100 pm, and others, in the nearest proximity of those hairs, having a reduced diameter (thin hair). Thus, by “reducing heterogeneity of the diameter”, is meant increasing the diameter of thin hair.

By “increasing the density”, is meant increasing the number of keratinic fibers, hairs or eyelash per square centimeter of skin or scalp.

### BRIEF DESCRIPTION OF THE DRAWINGS

The present invention is also illustrated, without being limited, by the following Figures:

FIG. 1: development of the size of the costo-vertebral organ (hereafter CVO) of the hamster under the effect of a topical application of testosterone (illustration of the “testosterone effect”).

D50 corresponds to the start of the daily applications of testosterone to the right CVO.

FIG. 2: inhibition of the “testosterone effect” by the polyunsaturated fatty acids, flavonoids and stilbenes, alone or in combination.

--- [symbol] --- controls
--♦-- black currant pips
--▴-- soja extract enriched with 40% content of isoflavones
--▪-- wine concentrate
---φ--- combination

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLES

### Example 1

### Demonstration of the Effect of a Topical Application of Testosterone to the Pilosebaceous Unit—In Vivo Test

1-A: Principle of the CVO Test:

The CVO of the hamster is a cutaneous region rich in pilosebaceous units.

The CVO test consists of determining the anti-androgenic action of different compounds on the CVO, i.e., of determining whether said compounds prevent the action of testosterone (Liao S et al., Arch Dermatol Res, 293(4), 200-206 (2001)).

1-B: Results of the Control Experiments: Observation of the “Testosterone Effect”:

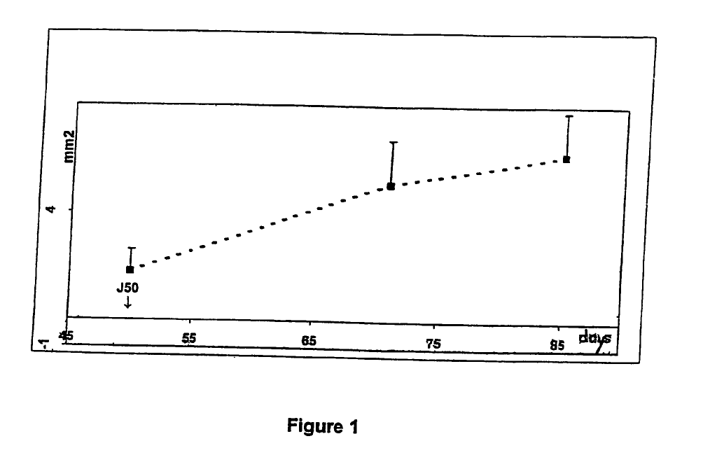

FIG. 1 shows the change in the size of the CVO in control animals which have not received any food supplement, in the case of topical testosterone application. More exactly, FIG. 1 shows the surface area difference between the right CVO, that has received testosterone, and the left CVO (negative control). Consequently, this figure illustrates the “testosterone effect”.

As from the fiftieth day (dotted arrow), testosterone was applied daily to the right CVO.

As FIG. 1 shows, testosterone induced over the course of time an increase in the size of the right CVO compared with that of the left CVO.

### Example 2

### Demonstration of the Inhibition of the “Testosterone Effect” by a Composition in Conformity with the Invention

2-A: Products Tested:

The following products were used in the context of the in vivo CVO test such as defined above. These products were tested as food supplements, alone or in combination.
(a) the natural aroma of red wine rich in resveratrol (stilbenes) and proanthocyanidines (flavonoids) in the proportion of 0.11 g in 43 g of food (18% of polyphenols);
(b) a soja extract containing 40% of isoflavonoids, in a proportion of 0.02 g of extract in 44 g of food (40% of isoflavones);
(c) black currant pip oil rich in γ-linolenic acid (polyunsaturated fatty acid 18:3 n-6) in a proportion of 10% by weight in the foodstuff;
(d) the combination of the above three products.

2-B: Results:

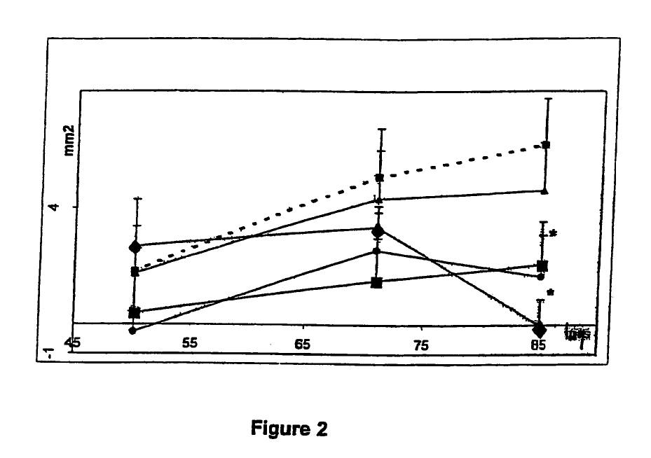

The product (b), namely a soja extract rich in isoflavonoids did not prevent the “testosterone effect” as observed in the context of the control experiments (Example 1-B), as illustrated in FIG. 2 by the “control” curves and “soja extract enriched with a 40% content of isoflavones”.

On the other hand, it will be seen from FIG. 2 that the products (a) and (c) as well as the combination of the products (a), (b) and (c), considerably reduced the difference in size between the right CVO and the left CVO.

The curve observed with the natural aroma of red wine (product (a)) retains, however, an upward slope, suggesting that the “testosterone effect” although advantageously slowed down, remained capable of a change with time contrary to the effect desired.

The curve observed with the product (c), corresponding to the oil containing polyunsaturated fatty acids, reflects a particularly strong, even sudden, inhibitory activity of the “testosterone effect” as from about day 20 of administration. The disadvantage linked to the use of the product (c) alone results from the fact that the difference in size between the right and left CVOs was increased compared with the controls (“controls” curve) during about the first five days of treatment.

Ultimately, only the combination of the products (a), (b) and (c), corresponding to a composition containing polyunsaturated fatty acids, flavonoids and stilbenes in conformity with the present invention, made it possible to observe the anticipated inhibition profile, namely, a considerable reduction of the “testosterone effect” as from the start of the treatment, followed by a progressive slowing down of said effect, in order to finally tend in the course of time towards a zero or practically zero effect.

In addition, no effect of the different compounds tested, alone or in combination, has been demonstrated on the sexual organs of the male animals. In fact, it was observed that the various products tested did not adversely affect the weight of the seminal vesicles and the prostate (results not shown). These observations indicate that a cosmetic composition in conformity with the invention ought not to present undesirable side effects of the compositions already known.

### Example 3

### Preparation of a Composition According to the Invention—Examples of Formulation

3-A: Formulation of the Sugar-Coated Type:

The quantities of the different compounds are indicated in mg per sugar-coated tablet.

Excipient of the Coating Core:

| | |
|---|---|
| microcrystalline cellulose | 70 |
| Encompress ™ | 60 |
| magnesium stearate | 3 |
| anhydrous colloidal silica | 1 |

Coating Agent:

| | |
|---|---|
| gum-lac | 5 |
| talc | 61 |
| sucrose | 250 |
| polyvidone | 6 |
| titanium dioxide | 0.3 |
| coloring agent | 5 |

Active Ingredient:

| | |
|---|---|
| black currant pip oil | 0.5 |
| red wine extract containing 18% polyphenols | 2 |
| soja extract enriched with 40% | 2 |
| content of isoflavones | |

A unit dose in the sense of the invention corresponds in kind to one sugar-coated tablet.

3-B: Formulation of the Plant or Animal Pelatine Capsule Type:

The quantities of the different compounds are indicated in mg per capsule.

Excipient:

| | |
|---|---|
| starch | 128 |
| magnesium stearate | 2.5 |

Active Ingredient:

| | |
|---|---|
| black currant pip oil | 200 |
| red wine extract containing 18% of polyphenols | 100 |
| soja extract enriched with 40% | 50 |
| content of isoflavones | |

The unit dose as defined in the preceding description corresponds to one capsule.

3-C: Formulations of the Unidose Gel Type:

The following quantities are expressed in % of the total weight of the final product.

According to this formulation, the unit dose is equivalent to 200 ml≈200 g

a) Unidose Gel 1:

Excipient:

| | |
|---|---|
| sugar syrup | 30 |
| maltodextrin | 17 |
| xanthan gum | 0 |
| sodium benzoate | 0.2 |
| water | qsp 100 |

Active Ingredient:

| | |
|---|---|
| black currant pip oil | 20 |
| red wine extract containing 18% of polyphenols | 10 |
| soja extract enriched with 40% content of isoflavones | 20 |

a) Unidose Gel 2:

Excipient:

| | |
|---|---|
| sugar syrup | 50 |
| maltodextrin | 17 |
| xanthan gum | 0.8 |
| sodium benzoate | 0.2 |
| water | qsp 100 |

Vitamins and Trace Elements:

| | |
|---|---|
| anti-oxidant complex | * |

Active Ingredient:

| | | |
|---|---|---|
| black currant pip oil | 10 | |
| red wine extract containing 18% of polyphenols | 12 | |
| soja extract enriched with 40% content of isoflavones | 24 | |
| * The anti-oxidant complex contains in 200 ml of gel: | | |
| vitamin C | 120 | mg |
| selenium | 100 | μg |
| vitamin E | 30 | mg |
| zinc | 20 | mg |
| p-carotene | 6 | mg |

3-D: Formulation of Capsule Type:

The following quantities are expressed in mg per capsule.

According to this formulation, a unit dose corresponds to a capsule.

a) Capsule 1:

Excipient:

| | |
|---|---|
| glycerol | 150 |
| magnesium stearate | 0.02 |
| water | qsp 900 |

Trace Element:

| | |
|---|---|
| zinc gluconate | 160 |

Active Ingredient:

| | |
|---|---|
| black currant pip oil | 300 |
| red wine extract containing 18% of polyphenols | 200 |
| soja extract enriched with 40% content of isoflavones | 200 |

b) Capsule 2:

Excipient:

| | |
|---|---|
| glycerol | 150 |
| magnesium stearate | 0.02 |
| water | qsp 900 |

Vitamins and Trace Elements:

| vitamin complex | * |
|---|---|
| zinc gluconate | 160 |

Active Ingredient:

| | | |
|---|---|---|
| black currant pip oil | 400 | |
| red wine extract containing 18% of polypheriols | 100 | |
| soja extract enriched with 40% content of isoflavones | 300 | |
| * The vitamin complex contains per capsule: | | |
| vitamin C | 60 | mg |
| selenium | 50 | μ |
| vitamin E | 15 | mg |
| zinc | 10 | mg |
| lycopene | 3 | mg |

3-E: Formulation of Soft Capsule Type:

The following quantities are expressed in mg per soft capsule.

Excipient:

| | |
|---|---|
| soja oil | 40 |
| wheat germ oil | 85 |
| soja lecithin | 25 |

Vitamins:

| | |
|---|---|
| natural tocopherols | 3 |
| vitamin C | 60 |

Active Ingredient:

| | |
|---|---|
| black currant pip oil | 200 |
| red wine extract containing 18% of polyphenols | 100 |
| soja extract enriched with 40% content of isoflavones | 100 |

Of this kind, a unit dose is equivalent to a soft capsule.

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. An orally administratable cosmetic composition suited for the prevention and/or treatment of functional disorders of the pilosebaceous structural unit of mammals, comprising a thus effective amount of immixture of polyunsaturated fatty acids, flavonoids and stilbenes, formulated into an orally administratable, physiologically acceptable medium therefor.

2. The orally administratable cosmetic composition as defined by claim 1, comprising an amount of said immixture of polyunsaturated fatty acids, flavonoids and stilbenes effective to combat the cutaneous activation of androgens over time.

3. The orally administratable cosmetic composition as defined by claim 1, comprising an amount of said immixture of polyunsaturated fatty acids, flavonoids and stilbenes effective for the treatment of oily and/or hyper-seborrheic skin and/or scalp, thin hair, hypertrichosis and/or alopecia in humans.

4. The orally administratable cosmetic composition as defined by claim 1, said polyunsaturated fatty acids and/or said flavonoids and/or said stilbenes being of natural origin.

5. The orally administratable cosmetic composition as defined by claim 1, said polyunsaturated fatty acids, said flavonoids and said stilbenes being of natural origin.

6. The orally administratable cosmetic composition as defined by claim 1, said polyunsaturated fatty acids being of the group n-6.

7. The orally administratable cosmetic composition as defined by claim 1, said polyunsaturated fatty acids comprising γ-linolenic acid.

8. The orally administratable cosmetic composition as defined by claim 1, said polyunsaturated fatty acids comprising an oil or an extract selected from the group consisting of black currant pip oil, evening primrose oil, borage oil, hemp oil, the extracts of spiruline and of Spirula maxima and S. platensis, and the oils of fishes and those derived from sea products, and black currant pip oil.

9. The orally administratable cosmetic composition as defined by claim 1, said flavonoids comprising isoflavonoids.

10. The orally administratable cosmetic composition as defined by claim 1, said flavonoids comprising isoflavones.

11. The orally administratable cosmetic composition as defined by claim 10, said isoflavones comprising daidzeine, genisteine or mixture thereof.

12. The orally administratable cosmetic composition as defined by claim 9, said flavonoids comprising plant extracts.

13. The orally administratable cosmetic composition as defined by claim 12, said flavonoids comprising the extracts of grape vines, tea, fruits, or soja.

14. The orally administratable cosmetic composition as defined by claim 1, said stilbenes comprising hydroxystilbenes.

15. The orally administratable cosmetic composition as defined by claim 14, said hydroxystilbenes comprising resveratrol or ester or glycosylated derivative thereof.

16. The orally administratable cosmetic composition as defined by claim 14, said stilbenes comprising extracts of the grape.

17. The orally administratable cosmetic composition as defined by claim 1, packaged in the form of unit doses for an administration of 1 to 6 units per day.

18. The orally administratable cosmetic composition as defined by claim 17, comprising from 5% to 60% by weight of polyunsaturated fatty acids per unit dose.

19. The orally administratable cosmetic composition as defined by claim 17, comprising from 5% to 60% by weight of flavonoids per unit dose.

20. The orally administratable cosmetic composition as defined by claim 17, comprising from 5% to 40% by weight of stilbenes per unit dose.

21. The orally administratable cosmetic composition as defined by claim 1, comprising at least one excipient suited for oral administration.

22. The orally administratable cosmetic composition as defined by claim 21, comprising one or more vitamins and/or trace elements.

23. The orally administratable cosmetic composition as defined by claim 1, formulated as a food supplement.

24. The orally administratable cosmetic composition as defined by claim 1, devoid of any Lotus extract.

25. The orally administratable cosmetic composition as defined by claim 1, devoid of DHEA or analogue thereof.

26. The orally administratable cosmetic composition as defined by claim 1, devoid of panthetin.

27. The orally administratable cosmetic composition as defined by claim 1, devoid of milk or extract thereof.

28. A regime or regimen for the prevention and/or treatment of a functional disorder of the pilosebaceous structural unit of a mammal, comprising orally administering to a mammalian organism in need of such treatment, a thus effective amount of immixture of polyunsaturated fatty acids, flavonoids and stilbenes, formulated into an orally administratable, physiologically acceptable medium therefor.

29. A regime or regimen for combating the cutaneous activation in a mammal of the androgens over time, comprising orally administering to a mammalian organism in need of such treatment, a thus effective amount of immixture of polyunsaturated fatty acids, flavonoids and stilbenes, formulated into an orally administratable, physiologically acceptable medium therefor.

30. A regime or regimen for treatment of oily and/or hyper-seborrheic skin and/or scalp, thin hair, hypertrichosis and/or alopecia afflicting a human, comprising orally administering to an individual in need of such treatment, a thus effective amount of immixture of polyunsaturated fatty acids, flavonoids and stilbenes, formulated into an orally administratable, physiologically acceptable medium therefor.

31. A regime or regimen for increasing the density of human keratinic and/or hair fibers and/or reducing the heterogeneity of the diameter thereof and/or improving growth thereof and/or preventing and/or reducing and/or delaying hair loss, comprising orally administering to an individual in need of such treatment, a thus effective amount of immixture of polyunsaturated fatty acids, flavonoids and stilbenes, formulated into an orally administratable, physiologically acceptable medium therefor.

32. The orally administratable cosmetic composition as defined by claim 1, formulated as a drinkable solution, a syrup, a tablet, a coated tablet, a gelatine capsule, an enriched foodstuff, a biscuit, a nutrient bar, or a powder.
